(19) United States
(12) Patent Application Publication
(10) Pub. No.: US 2021/0022963 A1
(43) Pub. Date: Jan. 28, 2021

(54) IMAGING ASSISTING DIAGNOSTIC DEVICE

(71) Applicant: Alaaeldin Soliman, Holmdel, NJ (US)

(72) Inventor: Alaaeldin Soliman, Holmdel, NJ (US)

(21) Appl. No.: 16/521,803

(22) Filed: Jul. 25, 2019

(51) Int. Cl. A61J 15/00; A61B 6/12

(52) U.S. Cl. CPC A61J 15/0007; A61M 2025/09166; A61B 6/12

(57) ABSTRACT

An image assisting diagnostic device includes a gastric tube; and a flexible cord removably attached to the gastric tube, wherein the flexible cord includes a first end having a probe tip; and a second end positioned at an opposite end from the first end, wherein the flexible cord includes a series of sections alternating between radiopaque material and substantially radiolucent material. The gastric tube includes a first diameter, and the flexible cord includes a second diameter, wherein the first diameter is different from the second diameter. The first diameter may be larger than the second diameter. The series of sections may be positioned between the first end and the second end of the flexible cord. The series of sections may be positioned closer to the first end than the second end. The probe tip may include radiopaque material.

### BACKGROUND

### Technical Field

The embodiments herein generally relate to medical devices, and more particularly to imaging techniques used with medical diagnostic devices.

### Description of the Related Art

Nasal gastric (NG) or oral gastric (OG) tubes are frequently inserted in patients, mostly for feeding or suctioning of stomach content. Confirming the proper placement of these tubes in the stomach is crucial to rule out incorrect placement in the lung or the esophagus. The confirmation of the proper placement is typically accomplished using an imaging study by performing an x-ray of the patient's abdominal cavity. NG and/or OG tubes are made of soft plastic or polyvinyl chloride (PVC) that does not appear in imaging studies.

In many occasions, especially in obese patients (i.e., patients with a body mass index (BMI) greater than 30), the abdominal fat adds a white shadow to the imaging study. This may result in guessing to find the tube tip and is subject to a large number of errors sometimes requiring re-placement of the NG/OB tube and another set of x-rays to confirm proper positioning of the tube. However, during medical emergencies these inefficiencies can mean the difference between life and death. Therefore, it is desirable to develop a new and improved technique for proper NG/OG placement in a patient.

### SUMMARY

In view of the foregoing, an embodiment herein provides an imaging assisting diagnostic device comprising a gastric tube; and a flexible cord insertable in the gastric tube, wherein the flexible cord comprises a probe tip; a handle end positioned opposite to the probe tip; and a plurality of rods spaced apart from one another and positioned in the flexible cord. The gastric tube may comprise a nasal gastric tube. The gastric tube may comprise an oral gastric tube. The plurality of rods may comprise radiopaque material. The flexible cord may comprise a section separating the plurality of rods from one another, and wherein the section comprises material that is non-detectable by x-ray imaging. The probe tip may comprise metal. The plurality of rods may comprise metal. The material may comprise plastic. The flexible cord may comprise plastic.

Another embodiment provides an image assisting diagnostic device comprising a gastric tube; and a flexible cord removably attached to the gastric tube, wherein the flexible cord comprises a first end comprising a probe tip; and a second end positioned at an opposite end from the first end, wherein the flexible cord comprises a series of sections alternating between radiopaque material and substantially radiolucent material. The gastric tube may comprise a first diameter, wherein the flexible cord comprises a second diameter, and wherein the first diameter is different from the second diameter. The first diameter may be larger than the second diameter. The series of sections may be positioned between the first end and the second end of the flexible cord. The series of sections may be positioned closer to the first end than the second end. The probe tip may comprise radiopaque material. The gastric tube may comprise a first gastric tube end; and a second gastric tube end, wherein, upon attachment of the flexible cord to the gastric tube, the first end of the flexible cord aligns with the first gastric tube end, and the second end of the flexible cord aligns with the second gastric tube end.

Another embodiment provides a method of confirming proper placement of a gastric tube inside a body, the method comprising inserting an imaging assisting diagnostic device inside the body, the device comprising: the gastric tube; and a flexible cord positioned in the gastric tube, wherein the flexible cord comprises a series of sections alternating between radiopaque material and substantially radiolucent material. The method further comprises performing x-ray imaging of the device, wherein the x-ray imaging detects the radiopaque material; and removing the flexible cord from the gastric tube and from the body. The method may comprise confirming the proper placement of the gastric tube based on the x-ray imaging. The method may comprise adjusting a position of the device in the body based on the x-ray imaging. The method may comprise performing additional x-ray imaging of the device after adjusting the position of the device to confirm the proper placement of the gastric tube inside the body.

These and other aspects of the embodiments herein will be better appreciated and understood when considered in conjunction with the following description and the accompanying drawings. It should be understood, however, that the following descriptions, while indicating exemplary embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 is a schematic diagram illustrating an imaging assisting diagnostic device, according to an embodiment herein;

FIG. 2 is a schematic diagram illustrating the imaging assisting diagnostic device of FIG. 1. with sections that are non-detectable by x-ray imaging, according to an embodiment herein;

FIG. 3 is a schematic diagram illustrating the imaging assisting diagnostic device of FIG. 1 with alternating sections of radiopaque material and radiolucent material, according to an embodiment herein;

FIG. 4 is a schematic diagram illustrating the imaging assisting diagnostic device of FIG. 1 depicting the different diameters of the gastric tube and flexible cord, respectively, according to an embodiment herein;

FIG. 5 is a schematic diagram illustrating the imaging assisting diagnostic device of FIG. 1 showing alignment of the gastric tube with the flexible cord, according to an embodiment herein;

FIG. 6A is a flow diagram illustrating a method of confirming the proper placement of a nasal or oral gastric tube inside a body/patient, according to an embodiment herein;

FIG. 6B is a flow diagram illustrating a method of confirming the proper placement of the nasal or oral gastric tube using x-ray imaging, according to an embodiment herein;

FIG. 6C is a flow diagram illustrating a method of adjusting a position of the imaging assisting diagnostic device in the body/patient, according to an embodiment herein; and

FIG. 6D is a flow diagram illustrating a method of performing additional x-ray imaging of the imaging assisting diagnostic device after adjusting the position of the imaging assisting diagnostic device, according to an embodiment herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

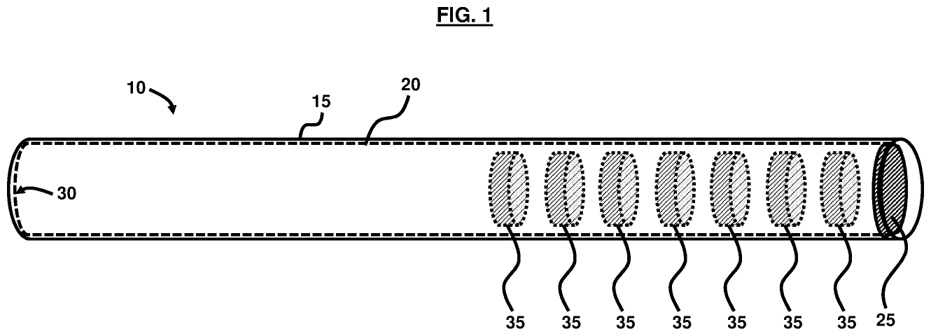

The embodiments herein provide a medical device that markedly enhances the tracking of a nasal or oral gastric tube that is inserted in a patient. Referring now to the drawings, and more particularly to FIGS. 1 through 6D, where similar reference characters denote corresponding features consistently throughout the figures, there are shown preferred embodiments. In the drawings, the size and relative sizes of components, layers, and regions, etc. may be exaggerated for clarity.

FIG. 1 illustrates an imaging assisting diagnostic device 10 comprising a gastric tube 15, and a flexible cord 20 insertable in the gastric tube 15. The gastric tube 15 may comprise a nasal gastric tube 15. Alternatively, the gastric tube 15 may comprise an oral gastric tube 15. The gastric tube 15 may comprise any suitable length necessary to be inserted into a patient's body from the mouth or nose and extending down into the patient's stomach. The gastric tube 15 is sufficiently flexible to permit insertion and proper orientation down the patient's esophagus into the stomach. Moreover, the gastric tube 15 is hollow and may be configured in any suitable manner typical of gastric tubes used in the industry, and the embodiments herein are not restricted to any particular configuration of the gastric tube 15. Additionally, the walls of the gastric tube 15 may have any suitable thickness to provide sufficient rigidity of the gastric tube 15 to be properly placed and retained inside the patient's body, yet sufficiently flexible for ease of maneuverability inside the patient's body. The gastric tube 15 may be substantially clear or translucent, according to an example.

The flexible cord 20 comprises a probe tip 25, a handle end 30 positioned opposite to the probe tip 25, and a plurality of rods 35 spaced apart from one another and positioned in the flexible cord 20. The flexible cord 20 is configured to be slidably inserted and removed from the gastric tube 15 multiple times, if necessary. The flexible cord 20 may comprise any suitable length necessary to be inserted into the gastric tube 15, which is inserted into the patient's body from the mouth or nose and extending down into the patient's stomach. The flexible cord 20 is sufficiently flexible to permit insertion and proper orientation into the gastric tube 15 for positioning down the patient's esophagus into the stomach. Moreover, the flexible cord 20 is hollow and may be configured in any suitable manner sufficient for insertion and retention into the gastric tube 15 until removal of the flexible cord 20 is desired in order to use the gastric tube 15 for feeding the patient. Furthermore, the embodiments herein are not restricted to any particular configuration of the flexible cord 20. Additionally, the walls of the flexible cord 20 may have any suitable thickness to provide sufficient rigidity of the flexible cord 20 to be properly placed and retained inside the gastric tube 15, yet sufficiently flexible for ease of maneuverability inside the gastric tube 15 and for ease of insertion and removal into/from the gastric tube 15. In an example, the flexible cord 20 may comprise plastic. Moreover, the flexible cord 20 may be substantially clear or translucent, according to an example.

The probe tip 25 of the flexible cord 20 may be formed from a different material than the flexible cord 20, according to an example. In an example, the probe tip 25 may comprise metal. Upon insertion of the flexible cord 20 into the gastric tube 15, the probe tip 25 may serve as the front end of the flexible cord 20 that permits the guiding of the flexible cord 20 down into the gastric tube 15. The handle end 30 of the flexible cord 20, which is positioned on an opposite end of the flexible cord 20 from the probe tip 25, may connect to a corresponding handle (not shown) used to hold the flexible cord 20 for insertion and guiding of the flexible cord 20 into the gastric tube 15 and for removal of the flexible cord 20 from the gastric tube 15. Any type of handle may be connected to the flexible cord 20 at the handle end 30, and the handle end 30 may be configured to removably attach a handle to the flexible cord 20.

The plurality of rods 35 may comprise radiopaque material. For example, the plurality of rods 35 may comprise metal. More particularly, the radiopaque material 60 may comprise mostly metal material. According to some examples, the radiopaque material 60 may comprise any of titanium, tungsten, barium sulfate, and zirconium oxide, among other suitable radiopaque materials. The plurality of rods 35 are spaced apart from one another and may comprise any suitable configuration; i.e., any suitable size, length, width, and thickness. Moreover, the plurality of rods 35 are embedded in the flexible cord 20 such that the plurality of rods 35 do not move in the flexible cord 20 once they are embedded in the flexible cord 20. In an example, the plurality of rods 35 may be press fit into position in the flexible cord 20. In another example, the plurality of rods 35 may be held in place (i.e., into position) by biosafe adhesive in the flexible cord 20. The embodiments herein are not restricted to any particular number of the plurality of rods 35. Additionally, the plurality of rods 35 may be solid or may be hollow. Furthermore, the plurality of rods 35 may be colored, according to an example. Additionally, the plurality of rods 35 may comprise multiple colors and each of the rods in the plurality of rods 35 may comprise a separate color from each other, according to various examples.

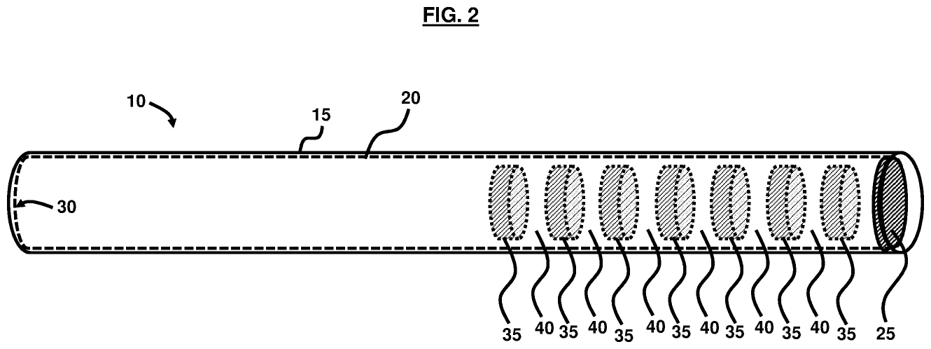

FIG. 2, with reference to FIG. 1, illustrates that the flexible cord 20 may comprise a section 40 separating the plurality of rods 35 from one another. Moreover, the section 40 comprises material that is non-detectable by x-ray imaging. For example, the material may be radiolucent. In an example, the material comprises plastic. The section 40 between each of the plurality of rods 35 serves as a spacing for the plurality of rods 35 with respect to each other. The size of each section 40 is not restricted to any particular length or configuration. Furthermore, each section 40 may comprise the same length as one another, in an example. Alternatively, the sections 40 may comprise different lengths as one another, in an example.

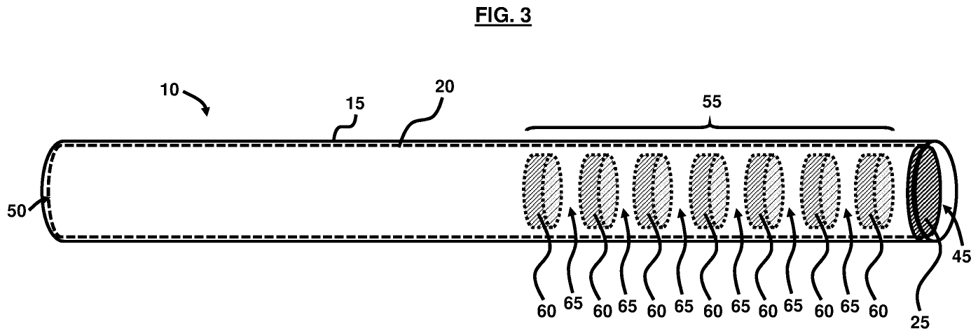

FIG. 3, with reference to FIGS. 1 and 2, illustrates an image assisting diagnostic device 10 comprising a gastric tube 15 and a flexible cord 20 removably attached to the gastric tube 15. The flexible cord 20 comprises a first end 45 comprising the probe tip 25. The flexible cord 20 comprises a second end 50 positioned at an opposite end from the first end 45. The second end 50 of the flexible cord 20 corresponds with the handle end 30 described above such that the second end 50 may be operatively connected to a handle (not shown) or other insertion device or tool to assist in maneuvering the flexible cord 20 into and out of the gastric tube 15. The flexible cord 20 comprises a series of sections 55 alternating between radiopaque material 60 and substantially radiolucent material 65. The radiopaque material 60 will appear in an x-ray image, while the substantially radiolucent material 65 will not appear in the x-ray image. In some examples, the radiopaque material 60 may comprise mostly metal materials, while the substantially radiolucent material 65 may comprise plastic materials.

The substantially radiolucent material 65 corresponds with the section 40 between the plurality of rods 35, as described above. The series of sections 55 are positioned between the first end 45 and the second end 50 of the flexible cord 20. In an example, the series of sections 55 may be positioned closer to the first end 45 than the second end 50. Furthermore, the embodiments herein are not restricted to any particular number or configuration of the alternating radiopaque material 60 and the substantially radiolucent material 65 in the series of sections 55. According to an example, the probe tip 25 may comprise radiopaque material, such as metal.

The substantially radiolucent material 65 may comprise plastic, according to an example. Furthermore, the size and length of the radiopaque material 60 in the flexible cord 20 and the size and length of the substantially radiolucent material 65 may be the same as one another, in an example. Moreover, the size and length of the radiopaque material 60 in the flexible cord 20 may be longer than the size and length of the substantially radiolucent material 65, in another example. Furthermore, the size and length of the radiopaque material 60 in the flexible cord 20 may be shorter than the size and length of the substantially radiolucent material 65, in still another example.

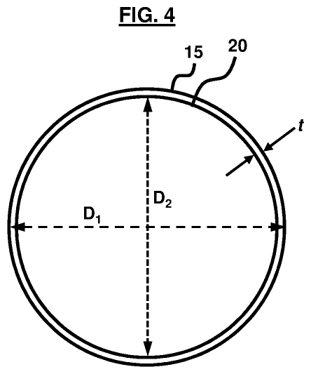

FIG. 4, with reference to FIGS. 1 through 3, illustrates that the gastric tube 15 comprises a first diameter D₁. Moreover, the flexible cord 20 comprises a second diameter D₂. Additionally, the first diameter D₁ is different from the second diameter D₂. In an example, the first diameter D₁ may be larger than the second diameter D₂ to permit the flexible cord 20 to be slidably engaged with respect to the gastric tube 15 such that the flexible cord 20 can be easily inserted and removed into/from the gastric tube 15. The gap between the flexible cord 20 and the gastric tube 15 is not limited to any particular gap thickness t so long as the flexible cord 20 can easily slide inside the gastric tube 15. Additionally, the size of the probe tip 25 is constrained by the size of the first diameter D₁ to further ensure that the entire flexible cord 20, including the probe tip 25, is removed from the gastric tube 15 once the gastric tube 15 is ready for being used to feed the patient. In this regard, according to an example, the probe tip 25 may comprise the same diameter as the second diameter D₂. In another example, the probe tip 25 may comprise a different diameter as the second diameter D₂, again so long as the diameter of the probe tip 25 is smaller than the first diameter D₁.

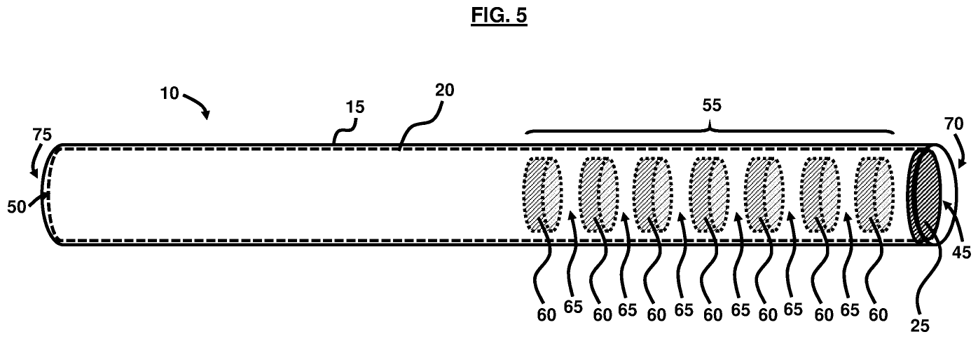

FIG. 5, with reference to FIGS. 1 through 4, illustrates that the gastric tube 15 comprises a first gastric tube end 70 and a second gastric tube end 75. Upon attachment of the flexible cord 20 to the gastric tube 15, the first end 45 of the flexible cord 20 aligns with the first gastric tube end 70, and the second end 50 of the flexible cord 20 aligns with the second gastric tube end 75. The first gastric tube end 70 is preferably closed to prevent the probe tip 25 of the flexible cord 20 from exiting the gastric tube 15 from the first gastric tube end 70. In an example, upon full insertion of the flexible cord 20 in the gastric tube 70, the probe tip 25 may abut the first gastric tube end 70. Conversely, according to an example, the second gastric tube end 75 is an open end of the gastric tube 15 to permit the flexible cord 20 to be inserted therein into the gastric tube 15, and subsequently removed from the gastric tube 15.

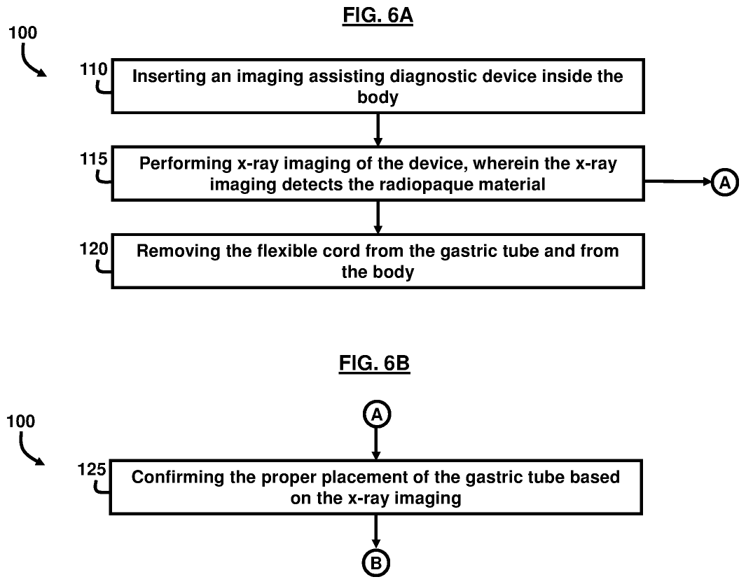

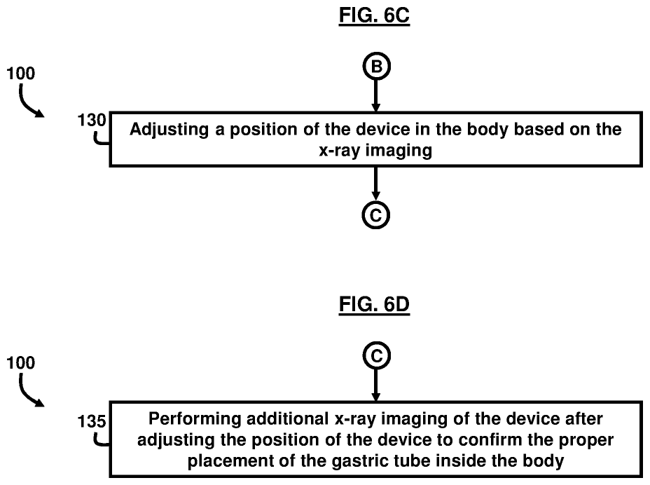

FIGS. 6A through 6D, with reference to FIGS. 1 through 5, illustrate a method 100 of confirming proper placement of a gastric tube 15 inside the body of a patient (not shown). As provided in FIG. 6A, the method 100 comprises inserting (110) an imaging assisting diagnostic device 10 inside the body of a patient. The imaging assisting diagnostic device 10 comprises a gastric tube 15 and a flexible cord 20, which is positioned in the gastric tube 15. The flexible cord 20 comprises a series of sections 55 alternating between radiopaque material 60 and substantially radiolucent material 65, as described above.

The method 100 further comprises performing (115) x-ray imaging of the imaging assisting diagnostic device 10 once the imaging assisting diagnostic device 10 has been inserted inside the body of the patient. The x-ray imaging detects the radiopaque material 60 associated with the plurality of rods 35 in the flexible cord 20. If the x-ray imaging reveals that the position of the plurality of rods 35 is aligned in a proper location inside the patient's stomach, then it is similarly determined that the gastric tube 15 is also in a proper location inside the patient's stomach since the flexible cord 20 with the plurality of rods 35 containing the radiopaque material 60 is inside the gastric tube 15 and is fully seated therein such that the probe tip 25 of the flexible cord 20 is abutting the first gastric tube end 70 of the gastric tube 15. The method 100 further comprises removing (120) the flexible cord 20 from the gastric tube 15 and from the body, upon confirming that the gastric tube 15 is properly positioned in the body of the patient. In order to remove the flexible cord 20 from the gastric tube 15, the flexible cord 20 may be pulled using a handle or other suitable mechanism connected to the handle end 30 of the flexible cord 20. However, because the flexible cord 20 slides with respect to the gastric tube 15, but does not move the gastric tube 15 or otherwise alter the position of the gastric tube 15 inside the patient's body, the gastric tube 15 remains in position in the patient's body while the flexible cord 20 (including the probe tip 25 and the plurality of rods 35) is completely removed. Accordingly, the gastric tube 15 that remains inside the patient's body is a hollow tube or device, which is ready to begin accepting fluids for feeding of the patient.

As shown in FIG. 6B, the method 100 may comprise confirming (125) the proper placement of the gastric tube 15 based on the x-ray imaging. In this regard, the x-ray imaging detects the position of the flexible cord 20 due to the detection of the plurality of rods 35 as well as the probe tip 25, which contain the radiopaque material 60. Accordingly, since the flexible cord 20 is fully seated in the gastric tube 15, then the proper position of the plurality of rods 35 indicates a corresponding proper placement of the gastric tube 15 inside the patient's body. Moreover, the full seating of the flexible cord 20 in the gastric tube 15 can be confirmed by the probe tip 25 abutting the first gastric tube end 70 of the gastric tube 15, which restricts further movement of the flexible cord 20 inside the gastric tube 15, or elsewhere, unless the flexible cord 20 is pulled and removed from the gastric tube 15 in a direction opposite to the first gastric tube end 70; i.e., out of the second gastric tube end 75 of the gastric tube 15.

As shown in FIG. 6C, the method 100 may comprise adjusting (130) a position of the imaging assisting diagnostic device 10 in the patient's body based on the x-ray imaging. Upon performing the x-ray imaging, if it is determined that the position of the plurality of rods 35 is improper or not preferable, then it can be inferred that the gastric tube 15 is similarly not properly positioned inside the patient's body. Accordingly, the gastric tube 15 with the flexible cord 20 inserted therein, may be further moved inside the patient's body until the medical clinician determines (i.e., based on medical judgment) that the gastric tube 15 is in a proper position in the patient's body. In this regard, the flexible cord 20 does not have to be removed from the gastric tube 15 until the x-ray imaging reveals a proper positioning of the plurality of rods 35, and accordingly, a corresponding proper positioning of the gastric tube 15. This saves time by allowing the gastric tube 15 containing the flexible cord 20 to be adjusted, if necessary, to a proper position inside the patient's body without having to remove the flexible cord 20 from the gastric tube 15 and then reinsert the flexible cord 20 into the gastric tube 15 in order to perform subsequent x-ray imaging, as further described below.

As shown in FIG. 6D, the method 100 may comprise performing (135) additional x-ray imaging of the imaging assisting diagnostic device 10 after adjusting the position of the imaging assisting diagnostic device 10 to confirm the proper placement of the gastric tube 15 inside the body. In this regard, the additional x-ray imaging detects the updated/revised position of the flexible cord 20 due to the detection of the plurality of rods 35 as well as the probe tip 25, which contain the radiopaque material 60. Again, since the flexible cord 20 is fully seated in the gastric tube 15, then the proper position of the plurality of rods 35 indicates a corresponding proper placement of the gastric tube 15 inside the patient's body. Moreover, the full seating of the flexible cord 20 in the gastric tube 15 can be confirmed by the probe tip 25 abutting the first gastric tube end 70 of the gastric tube 15, which restricts further movement of the flexible cord 20 inside the gastric tube 15, or elsewhere, unless the flexible cord 20 is pulled and removed from the gastric tube 15 in a direction opposite to the first gastric tube end 70; i.e., out of the second gastric tube end 75 of the gastric tube 15.

This process of adjusting the position of the gastric tube 15 with the inserted flexible cord 20 and performing x-ray imaging may occur as many times as necessary until the x-ray imaging reveals the proper placement of the plurality of rods 35, and therefore the proper placement of the gastric tube 15, inside the patient's body. Once, the x-ray imaging reveals the proper placement of the plurality of rods 35 and thus, the proper placement of the gastric tube 15, then the flexible cord 20 containing the plurality of rods 35 and probe tip 25 may be removed once and for all from the gastric tube 15, thereby permitting the hollow gastric tube 15 to receive fluids for nourishing the patient.

The foregoing description of the specific embodiments will so fully reveal the general nature of the embodiments herein that others may, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein may be practiced with modification within the spirit and scope of the appended claims.

What is claimed is:

1. An imaging assisting diagnostic device comprising:
a gastric tube; and
a flexible cord insertable in the gastric tube, wherein the flexible cord comprises:
a probe tip;
a handle end positioned opposite to the probe tip; and
a plurality of rods spaced apart from one another and positioned in the flexible cord.

2. The device of claim 1, wherein the gastric tube comprises a nasal gastric tube.

3. The device of claim 1, wherein the gastric tube comprises an oral gastric tube.

4. The device of claim 1, wherein the plurality of rods comprises radiopaque material.

5. The device of claim 1, wherein the flexible cord comprises a section separating the plurality of rods from one another, and wherein the section comprises material that is non-detectable by x-ray imaging.

6. The device of claim 1, wherein the probe tip comprises metal.

7. The device of claim 1, wherein the plurality of rods comprise metal.

8. The device of claim 5, wherein the material comprises plastic.

9. The device of claim 1, wherein the flexible cord comprises plastic.

10. An image assisting diagnostic device comprising:
a gastric tube; and
a flexible cord removably attached to the gastric tube, wherein the flexible cord comprises:
a first end comprising a probe tip; and
a second end positioned at an opposite end from the first end,
wherein the flexible cord comprises a series of sections alternating between radiopaque material and substantially radiolucent material.

11. The device of claim 10, wherein the gastric tube comprises a first diameter, wherein the flexible cord comprises a second diameter, and wherein the first diameter is different from the second diameter.

12. The device of claim 11, wherein the first diameter is larger than the second diameter.

13. The device of claim 10, wherein the series of sections are positioned between the first end and the second end of the flexible cord.

14. The device of claim 10, wherein the series of sections are positioned closer to the first end than the second end.

15. The device of claim 10, wherein the probe tip comprises radiopaque material.

16. The device of claim 10, wherein the gastric tube comprises:
a first gastric tube end; and
a second gastric tube end,
wherein, upon attachment of the flexible cord to the gastric tube, the first end of the flexible cord aligns with the first gastric tube end, and the second end of the flexible cord aligns with the second gastric tube end.

17. A method of confirming proper placement of a gastric tube inside a body, the method comprising:
inserting an imaging assisting diagnostic device inside the body, the device comprising:
the gastric tube; and
a flexible cord positioned in the gastric tube, wherein the flexible cord comprises a series of sections alternating between radiopaque material and substantially radiolucent material;
performing x-ray imaging of the device, wherein the x-ray imaging detects the radiopaque material; and
removing the flexible cord from the gastric tube and from the body.

18. The method of claim 17, comprising confirming the proper placement of the gastric tube based on the x-ray imaging.

19. The method of claim 17, comprising adjusting a position of the device in the body based on the x-ray imaging.

20. The method of claim 19, comprising performing additional x-ray imaging of the device after adjusting the position of the device to confirm the proper placement of the gastric tube inside the body.
